(19) United States
(12) Patent Application Publication
(10) Pub. No.: US 2006/0135440 A1
(43) Pub. Date: Jun. 22, 2006

(54) Use of ribose to alleviate rhabdomyolysis and the side effects of statin drugs

(76) Inventors: Mark C. Houston, Nashville, TN (US); John A. St. Cyr, Coon Rapids, MN (US)

Correspondence Address: Kathleen R. Terry, 13840 Johnson St. NE, Ham Lake, MN, 55304, US

(21) Appl. No.: 11/300,178

(22) Filed: Dec. 14, 2005

Related U.S. Application Data
(60) Provisional application No. 60/636,731, filed on Dec. 16, 2004.

(51) Int. Cl. A61K 31/70

(52) U.S. Cl. 514/23

(57) ABSTRACT

A method of alleviating the symptoms of rhabdomyolysis or the side effects of statin DRUG administration is disclosed. The method comprises the administration of D-ribose in doses of three to ten grams at least twice a day until the symptoms or side effects are alleviated.

### RELATED APPLICATIONS

This application claims priority from U.S. Provisional Application Ser. No. 60/636,731, filed Dec. 14, 2004.

### BACKGROUND OF THE INVENTION

Statins are a class of drugs developed to lower cholesterol in order to prevent the well known effects of high cholesterol, including heart attack, stroke and peripheral artery disease. Although they are very effective, statins induce a dose-related depletion of Co-Enzyme Q 10 in skeletal and cardiac muscle through inhibition of the geranylgeranyl PP pathway. This results in impaired production of ATP by the mitochondrial electron transport system, producing the symptoms of skeletal muscle fatigue and clinical myalgia in a significant number of patients taking statins in order to lower their cholesterol.

Some patients progress to life-threatening symptoms. One popular statin has been withdrawn from the market because clinical trials have shown a two-fold increase in heart attacks and strokes in patients taking the drug.

Rhabdomyolysis is a clinical and biochemical syndrome resulting from skeletal muscle injury with release of muscle cell contents into the circulation. The syndrome can result from many causes including a genetic defect, trauma, alcoholism, viral or bacterial infection, drugs and toxins. Whatever the cause, the pathogenesis appears to follow a final common pathway to an acute rise in cytosolic and mitochondrial calcium concentrations, leading eventually to muscle necrosis and cellular lysis. The noted reduction in cellular ATP levels and proposed mitochondrial damage appears to play a role in this disease state by producing a failure of Ca⁺⁺ ATPase activity, failure of Na⁺/K⁺ ATPase activity, and the generation of oxygen free radicals. Further, activation of degradative enzymes, such as phospholipase A₂ (PLA) and neutral proteases occurs, contributing to membrane phospholipid and myofibril damage. Loss of membrane integrity leads to the release of muscle proteins. Most of the complications of rhabdomyolysis may be due to the release of the muscle protein myoglobin into the circulation, subsequent accumulation of the myoglobin in the kidney tubules, leading to renal insufficiency.

Among the potential life-threatening complications are myoglobinuric acute renal failure, hypovolemia, hyperkalemia with potential cardiac arrest, disseminated intravascular coagulation and compartment syndrome. The overall mortality rate is about 5%, however, the morbidity and chronic health deficit is estimated to be much higher, that is, the condition is essentially incurable at this end stage of rhabdomyolysis. The primary diagnostic indicator of rhabdomyolysis is an elevated serum creatine phosphokinase to at least five times the normal value, beyond the levels diagnostic of myocardial infarction. Myoglobinuria is frequently seen. The patient presents with muscle pain, weakness, tenderness, malaise, fever, dark urine, nausea and vomiting. The pain may involve specific groups of muscles if caused by trauma, or may be generalized if due to other causes. Muscle pain can persist past the acute stage and may become chronic.

Of the complications, acute renal failure is seen in about 30-40% of cases of rhabdomyolysis. Other patients can suffer from reduced renal function. About 8 to 15% of all cases of acute renal failure are said to be caused by this syndrome. Treatment is palliative. The hypovolemia resulting from disseminated mucle edema may be reversed with saline injection, mannitol or Lasix administered to produce a urinary output of 200-300 cc/hour. Hemodialysis may be necessary in some cases. The drug Baycol is said to be responsible for more than 100 deaths worldwide from rhabdomyolysis.

Although these extreme side effects are rare, many patients suffer from persistent muscle pain. The beneficial effects of lowered cholesterol are not permanent; administration must be chronic in order to keep cholesterol levels down. The need remains to alleviate the side effects of statins. The need also remains to alleviate the symptoms of rhabdomyolysis from other causes.

### SUMMARY OF THE INVENTION

It is here disclosed that at least twice daily administration of two to ten, preferably four to eight, most preferably five grams of D-ribose alleviates the symptoms of rhabdomyolysis which include muscle pain, renal compromise, weakness, fatigue and/or stiffness.

It is also disclosed that at least twice daily administration of two to ten, preferably four to eight, most preferably five grams of D-ribose, alleviates the symptoms of those patients suffering milder side effects of statin administration which include muscle pain, weakness, fatigue and/or stiffness.

Alleviation is only partial for some patients at a twice daily administration of five grams of D-ribose; for those patients able to tolerate higher doses of D-ribose, up to ten grams per dose may achieve more alleviation. Administration of up to three to eight grams of D-ribose, preferably four times per day, may provide addition relief of symptoms. At these higher doses, it is advisable to co-administer ten grams of glucose or sucrose to avoid a hypoglycemic effect.

After an initial period at higher doses, the doses can be lowered to three to five grams of D-ribose, administered at least twice a day, preferably four times per day. When complete alleviation of symptoms has been achieved, administration of D-ribose may be discontinued or reduced unless symptoms recur.

### DETAILED DESCRIPTION OF THE INVENTION

### EXAMPLE 1

### Patient With Drug-Induced Rhabdomyolysis

A 49 year old male had a history of congestive heart failure secondary to myocardial infarction. He was started on a statin drug. Within three months he was diagnosed with rhabdomyolysis resulting in chronic renal failure and symptoms of extreme fatigue and muscle pain. The patient was placed on five grams of ribose, twice daily, after symptoms had persisted for about four months. Within one week, he reported that his muscle pain was gone and the fatigue greatly reduced. He stayed on the regimen for about three months. Upon cessation of ribose administration, the fatigue and pain slowly returned, but not as severe as had been previously experienced. He elected to return to the ribose regimen after a month and again experienced relief of symptoms.

### EXAMPLE 2

### Study Protocol for Pilot Study

Thirty patients aged 20 to 80 years of age presently taking statins and showing clinical statin-induced myalgias were randomized in a six-week placebo-controlled study of the effect of ribose on muscle pain. Fifteen patients were given five grams of ribose twice a day to determine the improvement in clinical symptoms, using an objective patient questionnaire and physician interview. The primary symptom reported was muscle pain, but other symptoms commonly reported included fatigue, poor sleep, a decrease in mental clarity and a reduced sense of well-being. Fifteen patients presently taking statins were given five grams of glucose twice a day as a control. All patients were given a complete history and physical exam. The patient's assessment of muscle pain, soreness or cramping was graded on a scale of one to five, five being the most severe.

Exclusions: Statin-induced myopathy with elevated CPJK over twice normal
Statin-induced hepatopathy with elevated LFTs over twice normal Pregnancy
Insulin-dependant diabetes
Diseases known to be associated with myalgias, such as fibromyalgia, PMR, CTD, genetic or acquired myopathies
New drugs or supplements during the study period

Inclusions:
Patients on any FDA-approved statin at recommended doses.
Constant use of statin dose and any other drugs for at least four weeks prior to study entry

Ribose or glucose were given as a pill or powder and compliance was monitored by pill count or powder measurement on each visit, weekly during the four week study period. A questionnaire was developed to validate the study results. Patient compliance, as summarized in Table I, was good.

TABLE I
Follow-up Distribution by Visit

| Visit | n (%) |
|---|---|
| Week 1 | 30 (100%) |
| Week 2 | 27 (90%) |
| Week 3 | 26 (87%) |
| Week 4 | 26 (87%) |
| Washout period | No ribose or glucose |
| | given |
| Week 6 | 23 (77%) |
| Week 6 | 18 (60%) |

All patients for whom the questionnaire was completed at the second week reported the presence of symptoms (muscle aching, pain, weakness, faitgue and/or stiffness) at each follow-up visit. A global assessment of symptom improvement during the course of the study was made for 13 ribose and 14 placebo patients. A total of 11 of the 13 (85%) ribose patients were classified as “Improved” compared with 3 of 14 (21%) of placebo patients. This result was highly significant (0=0.0018). The largest difference between the two groups occurred at the end of the test period, four weeks, where 10 (of 12 remaining; 83%) of the patients given ribose reported a change in symptoms compared with 5 (of 14 remaining, 36%) in the placebo group (p=0.02). Note that at the end of the test period, 87% of the patients were still attending their weekly visit. Following the four-week test period, patients were asked to continue their reporting. There was a drop off in compliance from 87% at the end of the four-week test period to 60% at week 6, the second week of the wash-out period.

The severity of symptoms were compared at each follow-up visit. Subjects were asked to rate the severity of symptoms on a scale from 1 to 5; 1=mild and 5=severe. Table II displays the results of averages of each group.

TABLE II
Severity of symptoms through follow-up

| | Ribose | Placebo |
|---|---|---|
| Visit | Mean (sd) | Mean (sd) |
| Week 1 | n = 15 | n = 15 |
| | 3.7 (0.8) | 3.0 (0.9) |
| Week 2 | n = 13 | n = 14 |
| | 3.9 (0.8) | 3.1 (0.9) |
| Week 3 | n = 12 | n = 14 |
| | 3.7 (0.8) | 3.0 (0.9) |
| Week 4 | n = 12 | n = 14 |
| | 3.6 (1.0) | 3.0 (0.9) |
| | Wash Out | |
| | Period | |
| Week 5 | n = 9 | n = 13 |
| | 3.4 (1.0) | 3.0 (0.9) |
| Week 6 | n = 8 | n = 10 |
| | 3.7 (0.8) | 3.0 (1.2) |

While, on the individual patient level, subjects in the ribose group tended to improve more during the course of the trial, average severity ratings were higher at each visit (see the global assessment rating above; 85% of the ribose group improved). This is particularly true at weeks 1 through 3, where the differences are statistically significant. Severity ratings stayed fairly constant for the placebo group (see global assessment rating above; 21% of the placebo group improved). This improvement is not apparent in the average rating of Table II.

Table III shows an individualized response to ribose or placebo, which shows more clearly that most patients did report general improvement of symptoms.

TABLE III

| | Ribose | Placebo |
|---|---|---|
| | (n = 12) | (n = 14) |
| More than 50% | 36% | 21% |
| improvement | | |
| Some | 73% | 43% |
| improvement | | |
| No improvement | 27% | 57% |
| or worse | | |

It can be noted that the ribose group, even though the inclusion of patients into one or the other group was randomized, reported more severity (3.7) at baseline than the placebo group (3.0). When a repeated measures analysis was performed, capturing the “average” response per patient per group and adjusted for the baseline severity difference (p=<0.0001), the ribose group has an average improvement of 0.10 units per week greater than the placebo group (p=0.35).

### EXAMPLE 3

### Study Protocol for Expanded Study

The pilot study of Example 2 used one level of ribose dosage, that is, five grams given twice a day. This dosage is well tolerated by most persons, and may be given more often, up to six times a day, without the gastrointestinal and hypoglycemic side effects long noted for oral ribose. Many persons may tolerate up to eight or ten grams per dosage. At this level, these patients should co-administer a source of sugars such as glucose or sucrose, in order to avoid hypoglycemia. Finally, a four week test study may be too short a period to show a beneficial effect in some patients, possibly the 27% with negative results in this study. A larger number of patients will be enrolled to study ribose at a dosage of five grams three or four times a day

The results reported in Example 2 show positive, but only partial relief of symptoms. Other studies will be performed to optimize and customize protocols to patients based on the size of patient, dosage and length of time on statin, and identity of the statin administered. It is believed that this information will result in protocols that give more benefit to a greater percentage of patients.

### EXAMPLE 4

### Ribose Administration in Related Conditions

There is a cluster of diseases of unknown etiology that share some common symptoms. Included in this cluster are chronic fatigue syndrome, fibromyalgia syndrome and statin-induced myalgia. The most prominent symptoms is pain in the muscles, accompanied by pain in connective tissue (fibromyalgia), sleep disturbance, lack of mental clarity and feelings of well-being. In addition to the patients of Example 1 and 2, who presented with statin-induced myalgia, a mixed cohort of patients having given a physician's diagnosis of chronic fatigue syndrome or fibromyalgia syndrome were given a supply of D-ribose for self administration. Each patient served as his or her control, the results being reported as pre- and post-ribose administration. Compliance was estimated by the amount of the ribose supply remaining. Those patients having administered at least one-half of the 280 grams supply were included in the following summary.

A total of 41 subjects were enrolled, of whom five were considered non-compliant and excluded from these analyses. Of the remaining 36 subjects, 78% were female, average age 48 years; 58% and 85% had been previously diagnosed with fibromyalgia and/or chronic fatigue syndrome. Twenty-nine subjects (81%) took a B-complex vitamin daily. The average length of time taking ribose was 25 days.

The primary outcome measure was the initial versus final visit Visual Analog Scale (VAS) score:
1. Visual Analog (well-being) Scale of 1-10 for five questions obtained before the first and after the last dose of ribose.
   (A) How is your energy? (1=near dead and 10+excellent)
   (B) How is your sleep? (1=no sleep and 10=8 hours of sleep a night without waking.
   (C) How is your mental clarity? (1=brain dead and 10=good clarity)
   (D) How bad is your pain? (1=very severe pain and 10=pain free)
   (F) How is your overall sense of well-being? (1=near dead and 10=excellent)

On taking the final dose, the patient was also asked in fill in: “much better, somewhat better, no change, somewhat worse and much worse.” The patients were also asked to comments on benefits or side effects. If they felt better, about how long did it take to feel better? Since the compliance was ascertained only by the amount of ribose remaining, the patients were asked to comment in more detail on their guesstimate of doses missed.

Table IV illustrates the average ratings for the pre- and post-ribose results.

TABLE IV
Pre- and post-ribose assessments of 36 patients

| Category | Pre- Mean (std) | Post- Mean (std) |
|---|---|---|
| Energy | 3.8 (1.1) | 5.5 (1.5) |
| Sleep | 4.8 (1.6) | 6.0 (1.9) |
| Mental | 4.9 (1.5) | 5.7 (1.7) |
| Clarity | | |
| Pain | 4.9 (2.3) | 5.6 (2.2) |
| Well-being | 4.3 (1.3) | 5.6 (1.5) |

In response to the general questions, a total of 23 of the 35 (65.7%) patients answering this question experienced at least some improvement during the course of this study, three patients reported that they felt no change to somewhat worse than at the beginning of the study.

Those skilled in the art may make insubstantial changes or additions to the above described invention. These changes and additions are within the scope of the appended claims.

We claim:

1. A method for alleviating the symptoms of rhabdomyolysis comprising the administration of an effective amount of ribose at least twice a day to a patient suffering from such symptoms until such symptoms are alleviated.

2. The method of claim 1 wherein the symptoms comprise muscle pain, renal compromise, weakness, fatigue and/or stiffness.

3. The method of claim 1 wherein the effective amount of ribose is three to ten grams.

4. The method of claim 1 wherein the effective amount of ribose is five to eight grams.

5. A method for alleviating the effects of statin administration comprising the administration of an effective amount of ribose at least twice a day to a patient suffering from such symptoms until such symptoms are alleviated.

6. The method of claim 5 wherein the symptoms comprise muscle pain, renal compromise, weakness, fatigue and/or stiffness.

7. The method of claim 5 wherein the effective amount of ribose is three to ten grams.

8. The method of claim 5 wherein the effective amount of ribose is five to eight grams.

9. A method of alleviating the side effects of statin administration comprising an initial administration of a first effective amount of ribose for at least twice a day to a patient suffering from such symptoms until such symptoms are alleviated and thereafter administering a reduced amount the dosage of ribose until or unless symptoms return.

10. The method of claim 9 wherein the first effective amount is five to ten grams and the reduced amount of ribose is zero to five grams.
